(19) United States
(12) Patent Application Publication
(10) Pub. No.: US 2012/0265214 A1
(43) Pub. Date: Oct. 18, 2012

(54) LOCKING MECHANISM FOR DEFLECTABLE INSTRUMENT SHAFTS AND METHOD OF USE

(76) Inventors: Nicholas J. Bender, Raleigh, NC (US); Carson Shellenberger, Raleigh, NC (US)

(21) Appl. No.: 13/418,340

(22) Filed: Mar. 12, 2012

Related U.S. Application Data
(60) Provisional application No. 61/451,211, filed on Mar. 10, 2011.

(51) Int. Cl. A61B 19/00

(52) U.S. Cl. 606/130

(57) ABSTRACT

An instrument having a deflectable section on an instrument shaft utilizes an actuator having first and second actuator sections moveable relative to one another to actuate a system of actuation elements such as pull cables. An actuator lock is operable to selectively fix the relative positions of the first and second actuator sections, thus setting the position of the instrument shaft's deflectable section.

This application claims the benefit of U.S. Provisional Application No. 61/451,211, filed Mar. 10, 2011, which is incorporated herein by reference.

### TECHNICAL FIELD OF THE INVENTION

The present invention relates generally to the field of medical instruments having steerable or deflectable shafts. In particular, the present invention relates to mechanisms for locking deflectable instrument shafts in a desired position.

### BACKGROUND

Surgery in the abdominal cavity is frequently performed using open laparoscopic procedures, in which multiple small incisions, trocar punctures, or ports are formed through the skin and underlying muscle and peritoneal tissue to gain access to the peritoneal site using the various instruments and scopes needed to complete the procedure. The peritoneal cavity is typically inflated using insufflation gas to expand the cavity, thus improving visualization and working space. Further developments have lead to systems allowing such procedures to be performed using only a single port.

In laparoscopic and single port surgery (“SPS”) procedures, it is useful to position a device within the incision to give sealed access to the operative space without loss of insufflation pressure. Some access devices suitable for use in SPS procedures and other laparoscopic procedures are described in co-pending U.S. application Ser. No. 11/804,063 ('063 application) filed May 17, 2007 and entitled SYSTEM AND METHOD FOR MULTI-INSTRUMENT SURGICAL ACCESS USING A SINGLE ACCESS PORT, U.S. application Ser. No.: 12/209,408 filed Sep. 12, 2008 and entitled MULTI-INSTRUMENT ACCESS DEVICES AND SYSTEMS, U.S. application Ser. No. 12/511,043, filed Jul. 28, 2009, entitled MULTI-INSTRUMENT ACCESS DEVICES AND SYSTEMS, U.S. application Ser. No. 12/649,307 , filed Dec. 29, 2009, entitled ACTIVE INSTRUMENT PORT SYSTEM FOR MINIMALLY-INVASIVE SURGICAL PROCEDURES, and U.S. application Ser. No. 12/846,788, filed Jul. 29, 2010 and entitled DEFLECTABLE INSTRUMENT PORTS. Each of the forgoing applications is incorporated herein by reference. The aforementioned patent applications describe instrument access tubes having deflectable distal ends. Flexible instruments disposed through the instrument access tubes are steered by actively deflecting the deflectable instrument delivery tubes. The present application describes mechanisms that may be used to retain or lock the deflectable instrument delivery tubes in a chosen position.

### BRIEF DESCRIPTION OF THE DRAWINGS

FIG. 1 is a perspective view showing an instrument port utilizing the actuator lock of the first embodiment;

FIG. 2 is similar to FIG. 1 but shows the actuator engaged to deflect the instrument delivery tube;

FIG. 3 is a partially exploded view of the actuator of the instrument port of FIG. 1, showing the actuator lock separated from the actuator;

FIG. 4 is a longitudinal cross-section view of the actuator and actuator lock of FIG. 1;

FIG. 5 is a perspective view of the actuator of an instrument port utilizing a second embodiment of an actuator lock;

FIG. 6 is an enlarged view of the portion of the actuator lock encircled in FIG. 5;

FIG. 7 is an exploded perspective view of the actuator of FIG. 5;

### DETAILED DESCRIPTION

The present invention describes a locking mechanism suitable for use in conjunction with a steerable or deflectable medical instrument shaft for locking the shaft in a chosen position. The application describes the locking mechanism in the context of an embodiment for which the steerable/deflectable shaft is that of an instrument port that function as a deflectable conduit through which passively flexible medical instruments are passed into the body. The described ports include actuators positioned outside the body that allow active deflection of the distal ends of the ports, and thus the distal ends of the flexible instruments passed through them. It should be understood, however, that while the disclosed instrument shafts are those of instrument ports, the actuators and associated locking mechanism may instead be incorporated into other types of medical devices, such as instruments having integrated end effectors.

A deflectable instrument port 10 is shown in FIG. 1. The port has an elongate instrument delivery tube 16 having a flexible, deflectable, distal section 20. The tube 16 may be flexible along its full length, or it may have a rigid proximal section proximal to the flexible distal section 20. An actuator 22 controls deflection of the flexible distal section 20 of the instrument delivery tube 16.

A plurality of actuation elements 18 (which in this description may also be referred to as pull wires or cables but which may take alternate forms) extend through the instrument delivery tube 16 and are anchored near the distal end. In the preferred embodiment, each instrument delivery tube has four such wires arranged at 90 degree intervals. Other embodiments can utilize different numbers of pullwires, such as three pullwires equally spaced around the instrument delivery tube 16. The above-referenced applications which are incorporated by reference describe features that may be used for the instrument delivery tube 16, the routing and anchoring of the actuation elements 18, and associated features. The actuation elements 18 are coupled to the actuator 22 (FIG. 1), which selectively tensions the actuation elements to deflect the distal section 20.

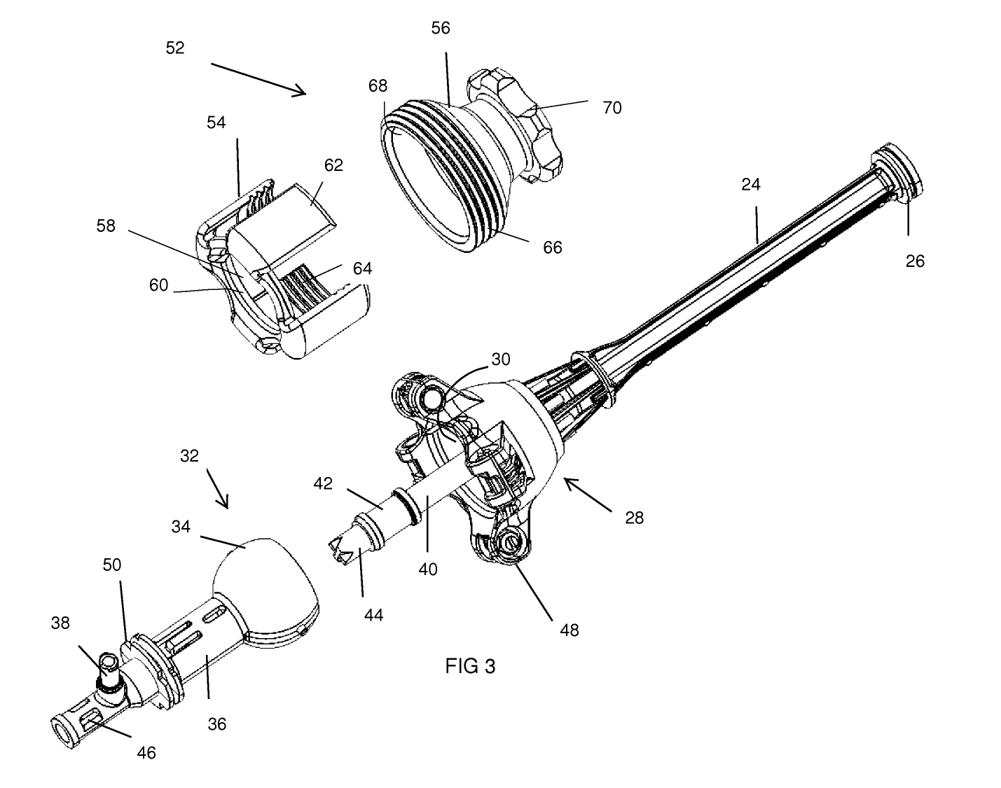

The actuator employs a ball and socket type arrangement to tension the actuation elements 18. FIG. 3 shows details of the actuator 22, which may includes features similar to those shown and described in U.S. application Ser. Nos. 12/209,408, filed Sep. 12, 2008, 12/511,043, filed Jul. 28, 2009, and 12/846,788, filed Jul. 29, 2010. The instrument delivery tube 16 and the actuation elements 18 are not shown in FIG. 3.

In use, the distal end of a flexible instrument to be deployed into the body cavity via the port 10 is inserted into a control tube 24 on the actuator 22 and then advanced into and through the lumen of the instrument delivery tube 16—so that the instrument's operative end extends out of the delivery tube 16 into the body cavity. Manipulating the proximal end or handle of the instrument in turn moves the control tube 24 and engages the actuator to deflect the instrument delivery tube 16 at distal section 20. This deflects the operative end of the instrument within the body cavity.

Referring to FIG. 3, each actuator 22 includes the control tube 24 and a proximal entry port 26 for receiving a medical instrument. Entry port 26 includes a septum seal for sealing against the shaft of an instrument passed through it. The control tube 24 may have an inner tubular lining, preferably formed of a lubricious material such as PTFE or other suitable material so as to allow instruments inserted through the actuator to slide with ease. A proximal actuator portion 28 is coupled to the distal end of the control tube 24. The proximal actuator portion 28 has a distally-facing socket 30. A distal actuator portion 32 includes a ball section 34 having a partially spherical surface partially disposed within the distally-facing socket 30 of the proximal actuator portion. The ball section further includes a tubular housing 36 that extends distally from the ball and is coupled to the instrument delivery tube 16. A side opening in the tubular housing 36 is fluidly coupled to a luer port 38. The luer port allows insufflation gas or irrigation fluid to be selectively introduced through the instrument delivery tube and into the body cavity.

A tube 40 extends through the proximal and distal actuator portions 28, 32 and has its distal end secured within the tubular housing 36 by a fitting 42. A valve 44, which may be a cross-slit duck bill valve, is disposed within the tubular housing 36. The valve functions to seal the actuator against loss of inflation pressure when no instruments are positioned through it.

The actuation elements 18 (not shown in FIG. 3) exit the proximal end of the instrument delivery tube 16 and extend out of the housing 36 through slots 46. The proximal ends of the actuation elements are coupled to the proximal actuator portion 28 at anchor points on radial members 48. Guides 50 help to maintain the alignment of the actuation elements 18 outside the housing 36.

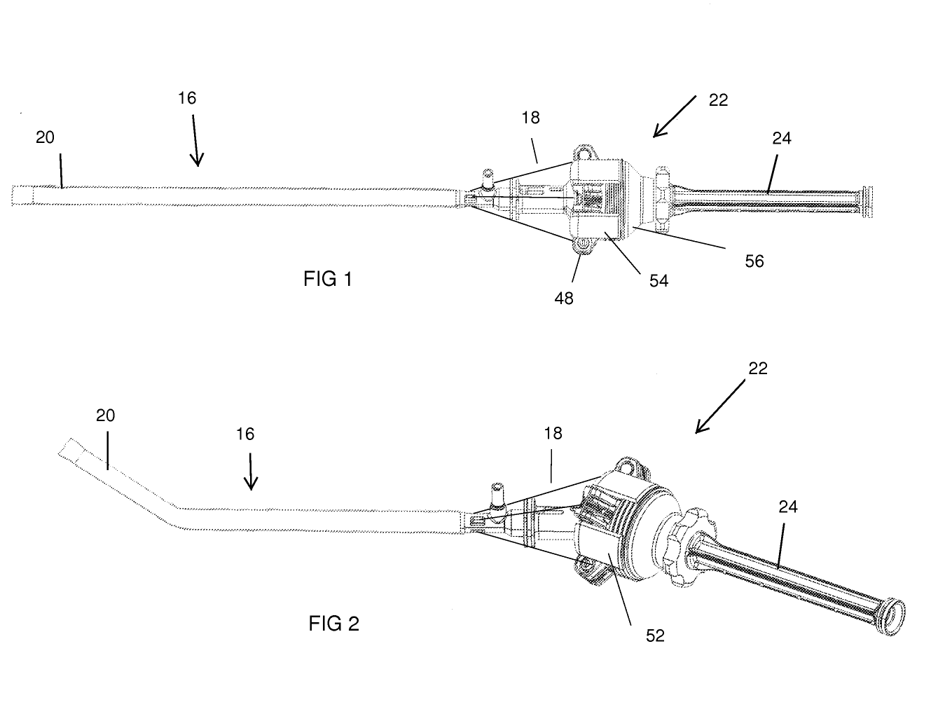

During use of the actuation system, the shaft of an instrument to be deflected using the port 10 extends through the control tube 26, proximal actuator portion 28, distal actuator portion 32 etc. and through the instrument delivery tube 16 such that its operative end is disposed within the body cavity. A suitable instrument will have a rigid proximal section that will be disposed within or otherwise in contact with the control tube 26, and a flexible distal section. To articulate the distal end of the instrument, the surgeon moves the handle of that instrument, causing the control tube 24 to move with it. The proximal actuator portion 38 will move over the ball surface of the distal actuator portion 28, thus tensioning the pullwires in accordance with the angular position of the proximal actuator portion relative to the longitudinal axis of the distal actuator portion. The distal portion of the instrument will deflect accordingly as a result of the action of the actuator on the pullwires of the instrument delivery tube. Thus if it is desired to raise the distal end of the instrument, the user will lower the handle, moving the proximal actuator portion downwardly over the ball surface. This will thus, apply tension to the upper pullwire 18, causing upward deflection of the instrument delivery tube as well as the distal end of the instrument. Lateral movement of the instrument shaft to the right will tension the corresponding side pullwire to cause the distal portion of the instrument delivery tube to bend to the left. FIG. 2 shows the control tube 24 moved laterally and the corresponding deflection of the instrument delivery tube 16.

The actuator system allows combinations of vertical and lateral deflection, giving 360° deflection to the instrument delivery tube. In other embodiments, the pullwires may be routed such that the movement of the distal section 20 matches that of the control tube 24 (e.g. lifting the control tube lifts the distal end of the instrument delivery tube 16 and instrument).

The port may include a mount for coupling the device to a support/stabilization arm coupled to an operating table, cart, operating room ceiling, or other operating room fixture. One example of a stabilization arm suitable for this purpose is shown and described in co-pending application Ser. No. 12/846,788, filed Jul. 29, 2010, incorporated herein by reference.

Actuator lock 52 is positioned to allow the user to selectively restrain the proximal actuator portion 28 so as to temporarily fix the relative positions of the proximal actuator portion 28 and the distal actuator portion 32—thus temporarily retaining the deflectable section 20 of the instrument tube at its chosen deflected (or straight) orientation. This may be accomplished using a clamp positioned to clamp the ball and socket between clamp sections when the relative distance between the clamp sections is decreased by the user, thus frictionally engaging the ball and socket to one another.

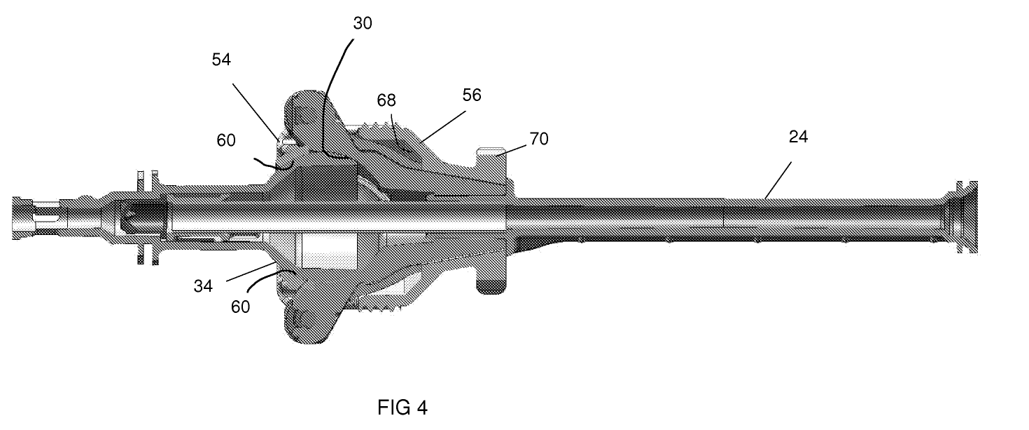

In FIG. 3, the actuator lock 52 is shown separate from the other parts of the actuator 22. The actuator lock 52 includes first and second clamp halves 54, 56. The first clamp half 54 has a distal side with an opening 58 encircling the ball 34 of the distal gimbal section 32. The edges 60 surrounding the opening have a curved or canted surface positioned in proximity to the distally adjacent portions of the ball surface as shown in FIG. 4. Members 62 extend proximally from the distal side of the clamp half 54 and are positioned such that their inner surfaces collectively lie in a generally cylindrical arrangement. A thread pattern 64 is formed on the inner surfaces of the members 62, and the circumferential spaces between the members 62 are positioned to receive the proximal actuator portion's radial members 48 (see FIG. 1).

Clamp half 56 has a distally-facing receptacle or opening 68 which seats over a portion of the proximal actuator portion 28. A circumferential thread pattern 66 on the outer surface of clamp half 56 engages with the thread pattern 64 of clamp half 54. Clamp half 56 may also include a proximal knob 70. When the user rotates the clamp half 56 (e.g. using the knob 70) in a clockwise direction, the clamp half 56 advances distally relative to the clamp half 54 due to the engagement of the threads 64, 66. This distal advancement of the clamp half 54 presses the surface of the gimbal socket 30 into firm contact with the outer surface of the ball section 34, causing the proximal and distal actuator portions 28, 32 to frictionally engage. This frictional engagement retains the orientation of the proximal actuator portion 28 relative to the distal actuator portion, allowing the user to temporarily fix the deflectable distal section 20 in a chosen orientation. To allow further deflection or straightening of the distal section 20, clamp half 56 is rotated in a counterclockwise direction, moving the clamp half 56 proximally relative to the clamp half 54, releasing the frictional engagement between the proximal and distal actuator portions.

The clamp half 56 or knob 70 may be coupled with the control tube 24 such that axial rotation of the control tube 24 will cause rotation of the clamp half 56. This arrangement facilitates single handed use of the actuator 22 and actuator lock by allowing the user to slide his/her hand forward from the instrument to the control tube 24 and to then rotate the control tube 24 in order to engage/disengage the actuator lock.

It should be noted that during use the lock may be employed to frictionally engage the ball and socket by an amount that will maintain the relative positions of the ball and socket even if the user removes his/her hand from the control tube 24 or corresponding instrument handle (and thus set the deflected (or undeflected) position of the distal section 20 of the tube 16) but that will allow relative movement between the ball and socket in response to the user's application of force on the control tube 24 (i.e. an amount greater than needed to steer deflectable section 20 when the lock is disengaged). This feature allows the user to make slight adjustments to the position of the deflectable section 20, and to be able to then remove his/her hand from the control tube 24 or instrument handle and have the selected position of the deflectable section 20 remain. For example a user might, with the lock disengaged, manipulate the instrument handle or control tube 24 to position the distal end of the instrument in a first desired position, then engage the lock to retain that first position. With the lock so engaged, the user may apply force to the control tube 24 or user handle to move the socket relative to the ball and to thus change the position of the instrument tip to a second position, and then remove his/her hand from the control tube 24 or instrument handle such that the instrument tip will remain in the second position without the need to disengage and reengage the lock.

A port of the type shown herein may be beneficially used for tissue retraction during a surgical procedure. In particular, a surgical retractor such as a grasper may be inserted into control tube 24 and advanced such that the grasping end effector extends from the deflectable distal section 20. The tube 16 is inserted through an incision (either directly or through a port disposed within the incision. The port 10 is mounted to a stabilization arm. The handle of the grasper is manipulated to move the control tube, thus steering the end effector into the desired position. Tissue is engaged using the grasper, and the grasper handle/control tube are manipulated to retract the tissue. With the tissue in the retracted position, the gimbal lock is engaged, thus maintaining tissue retraction. Adjustments to the retraction may be made by adjusting the position of the control tube 24 by moving the control tube or instrument handle (either by disengaging the lock or by resisting the frictional engagement of the lock as described in the preceding paragraph), or by adjusting the orientation of the port 10 relative the stabilization arm and/or by adjusting the orientation of the stabilization arm relative to the patient.

While in the illustrated first embodiment, a thread connection between the clamp halves 54, 56 is used to decrease the relative distance between the clamp sections so as to frictionally engaging the ball and socket to one another, alternate mechanisms may instead be used. For example, a cam may be positioned to cam one or both of the clamp sections towards the other. As another example, a ratchet feature may be used to advance or both of the clamp sections towards the other.

Moreover, while the disclosed embodiment employs friction to restriction relative movement between the ball and socket, other embodiments might employ members such as teeth, spikes or serrate edges to bite into the surface of the ball such that it will resist rotation relative to the socket.

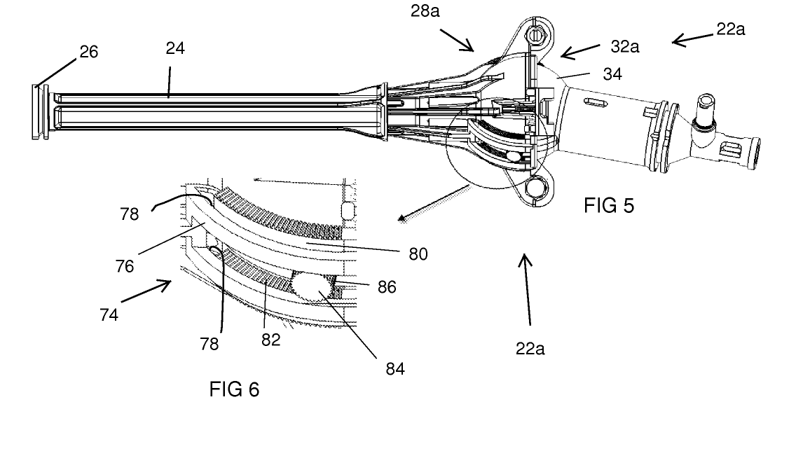

FIGS. 5 through 7 illustrate an actuator 22a for a deflectable instrument port utilizing an alternative actuator lock. Many features of the FIG. 5-7 instrument port are similar to those of the instrument port of the first embodiment and will not be described again here.

The proximal actuator portion 28a of the actuator 22a includes a pair of supports 74. In each of FIGS. 5-7, one such support is shown. The other support is positioned 180° from the one that is visible, i.e. on the opposite side of the distally-facing socket 30. The support 74 includes a pair of spaced apart rails 76 curved in parallel to the spherical surface of the ball 34. Each rail 76 includes an elongate slot 78. Curved locking plates 80 are disposed in each slot 78. Ridges 82 are formed in the edges of each locking plate 80. The locking plates are slidable within their respective slots in directions towards and away from another.

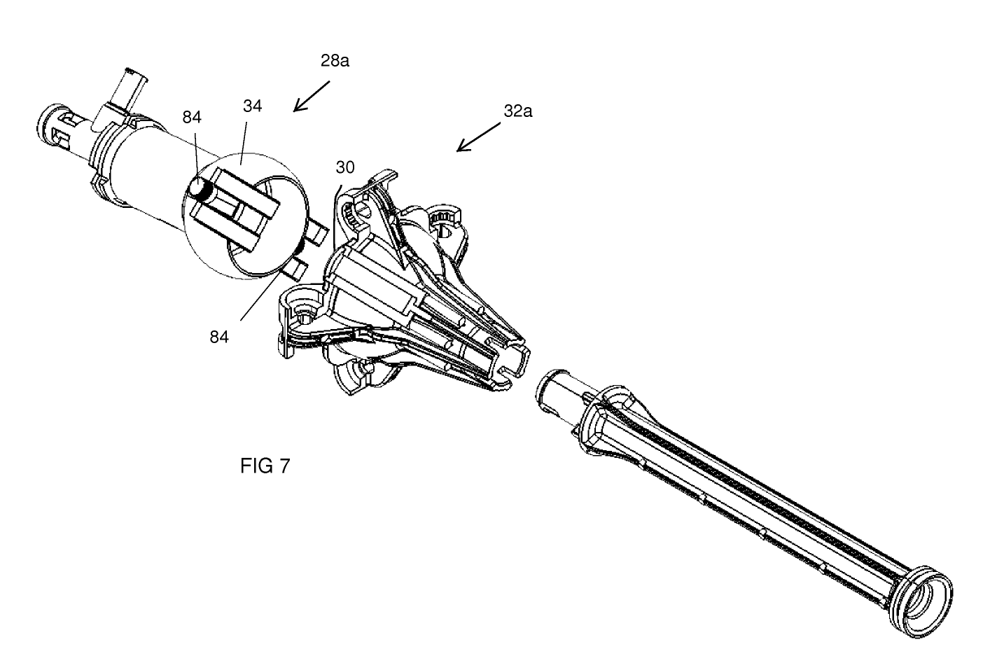

As best shown in FIG. 7, the ball 34 of the distal actuator portion 32a includes a pair of posts 84 extending from the surface of the ball 34. Each post 84 has circumferential ridges 86 proportioned to engage with the ridges 82 of the locking plates 80. It should be noted that while the ridges 82, 86 are used in the illustrated embodiment, the posts 84 and locking plates 80 may be engageable with different features other than ridges.

When the actuator 22a is assembled, the post 84 is positioned between the rails 76. The locking plates are disposed at a sufficient lateral distance from one another so they can move without contacting the post 84 when the proximal actuator portion 28a is moved over the surface of the ball 34.

During use of the second embodiment, the user steers/deflects the distal end of an instrument placed through the instrument port in the same manner as described with regard to the first embodiment. In particular, the use manipulates the proximal end of the instrument, causing movement of the control tube 24 and thus movement of the proximal actuator portion 28a relative to the distal actuator portion 32a, thereby engaging the pull elements and steering the distal portion of the instrument tube 16 (not shown in FIGS. 5-7 but see FIG. 1). To retain the instrument tube 16 in a desired position, the user will move each pair of locking plates 80 towards the post disposed between them by sliding the locking plates 80 within their respective slots (this may be achieved, for example, by pinching each pair of locking plates 80 between the user's thumb and forefinger). The locking plate ridges 82 engage with the ridges 86 on the post 84, restraining the proximal actuator portion 28a against movement relative to the distal actuator portion 32a.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Moreover, features of the various disclosed embodiments may be combined in various ways to produce various additional embodiments.

Any and all patents, patent applications and printed publications referred to above, including for purposes of priority, are incorporated herein by reference.

We claim:

1. A method of performing a surgical procedure, comprising the steps of;
providing an instrument port comprising an elongate tube comprising a rigid section having a fixed shape and a deflectable section distal to the rigid section, an actuator comprising a ball member and a socket member coupled to the rigid section of the elongate tube, a plurality of actuation elements extending between the actuator and the deflectable section, and a mount coupled to the elongate tube;
forming an incision in body tissue;
inserting the distal end of the elongate tube through the incision and positioning the instrument port such that the rigid section traverses the incision;
positioning the instrument port in a desired orientation;
coupling the mount to an operating room fixture to retain the instrument port in the desired orientation;
inserting an instrument through the actuator and the elongate tube such that a distal end of the instrument is distal to the deflectable section and such that a proximal end of the instrument is in contact with the actuator; and
manipulating the proximal end of the instrument, causing one of the ball and socket members to move relative to the other of the ball and socket members placing the actuator in a first actuator position, thereby engaging the actuation elements and deflecting the deflectable section of the elongate tube to a first position; and
frictionally engaging the ball and socket members to restrain the actuator in the first actuator position, thereby retaining the deflectable section in the first position.

2. An instrument port comprising:
an elongate tube having a lumen, the elongate tube comprising a deflectable section;
an actuator coupled to the proximal end of the elongate tube and including a proximal actuator portion moveable relative to a distal actuator portion, wherein the actuator includes an instrument pathway in communication with the lumen, the instrument pathway positioned such that a distal end of a medical instrument may be inserted through the instrument pathway and the lumen and out the distal end of the lumen into a body cavity;
a plurality of actuation elements extending between the proximal actuator portion and the deflectable section, whereby manipulation of a proximal end of a medical instrument disposed in the instrument pathway and lumen engages the actuation elements to deflect the deflectable section; and
an actuator lock engageable to restrain movement of the proximal actuator portion relative to the distal actuator portion, thereby selectively retaining the deflectable section in a predetermined position.

3. The portion of claim 2, wherein the actuator lock includes first and second clamp sections positioned to clamp the proximal and distal actuator portions, thus frictionally engaging the proximal clamp section against the distal clamp section.
